(19) United States
(12) Patent Application Publication
(10) Pub. No.: US 2009/0269288 A1
(43) Pub. Date: Oct. 29, 2009

(54) Black pearl toothpaste

(76) Inventor: Zoya Lavrova, Brooklyn, NY (US)

Correspondence Address: Aleksandr Smushkovich, P.O. Box 140505, Brooklyn, NY, 11214, US

(21) Appl. No.: 12/381,940

(22) Filed: Mar. 18, 2009

Related U.S. Application Data
(60) Provisional application No. 61/125,260, filed on Apr. 24, 2008.

(51) Int. Cl. A61K 8/46; A61Q 11/00

(52) U.S. Cl. 424/56

(57) ABSTRACT

A proposed toothpaste is disclosed providing a synergistic composition for toothpaste to prevent decay, to prevent and control dental plague and gingivitis, and to suppress growth of wide spectrum of micro organisms that cause periodontal disease, while not exhibiting any harmful particular side effect. It allows absorbing and removing odor components out of the oral cavity for a long period of time, removing stain from the teeth, and polishing them. The toothpaste comprises: Sodium Monofluorophosphate, Glycerin, Water, Calcium Carbonate, Xylitol-70%, Sodium Dodecyl Sulfate, Sodium Saccharin, Sodium Bicarbonate, Sodium Methylparaben, Xanthan Gum (E-415), Cellulose Gum (E-466), Hydrated Silica, Titanium Dioxide (E-171), Peppermint Oil, Commiphora Myrrh Extract, Menthol, Charcoal, Grape Seed Oil, and Extract of Brazilian Acai Berry. Particular amounts of weight units for preferred embodiments are provided, as well as a sample method of preparation the toothpaste.

### CROSS REFERENCE TO RELATED APPLICATIONS

The present U.S. utility patent application claims the benefit under 35 USC 119 (e) of a U.S. provisional patent application No. 61/125,260 filed on 24 Apr. 2008, whose disclosure is hereby incorporated in its entirety by reference. This application disclosure also contains newly disclosed ramifications that do not claim the aforesaid benefit.

### FIELD OF THE INVENTION

The present invention relates to toothpaste products, particularly to those capable of preventing decay, preventing and controlling dental plague and gingivitis, suppressing the growth of wide spectrum of micro organisms that cause periodontal disease, etc.

### BACKGROUND OF THE INVENTION AND RELEVANT ART

Periodontal disease is one of the most prevalent dental chronic diseases. Children as young as 5 years of age can already have the disease. By the age of 35 three out of four people are affected, and by the age of 65, as estimated, 98% of Americans have the periodontal disease. Before the onset of disease, healthy pink Gingiva, or gum tissue, covers the neck of the tooth and serves to hold the tooth in place and to prevent microbial materials from entering the jaw bone or the tooth itself. The area between the tooth enamel and the Gingiva is called a Gingival Crevice. When the gingivitis occurs, the gums are constantly exposed to infection. Gingivitis typically develops when large masses of bacteria accumulate in the gingival crevice; the first sign of gingivitis is gum bleeding. This inflammatory condition is reversible. Gum disease occurs when the gingival crevice between the tooth and the gum is more than 3 mm deep. As gingivitis progresses, the tissue surrounding the teeth is destroyed and the supporting collagen fibers degenerate. That leaves abnormally deep crevice or pockets adjacent to the teeth due to the loss of gum attachment and bone loss. Chronic periodontitis is characterized by chronic plague and calculus accumulation, gingival inflammation and loss of attachment and bone. Usually it is a slow painless progressing process.

Gum disease and the caries infection is a very complex biofilm infection. Strains of bacteria appear at this stage, and even some strains of yeast occur. These bacteria have the ability to metabolize, using ferments. The carbohydrates and the sugars that we ingest also feed the oral bacteria that produce lactic and acetic acids as waste products. These acids are responsible for eating away the hard coating enamel and causing demineralization which results in tooth decay.

Practicing good oral hygiene is critical in the prevention of tooth decay and periodontal gum disease. Areas that are particularly problematic for cleaning with a toothbrush are near the gum line (beneath the lower teeth and above the upper teeth) and in interproximal (between) tooth surfaces. Some of the bacteria are presented in the form of a clear (almost invisible) sticky film, plaque, which must be mechanically removed by brushing and flossing.

The plaque forms at all ages, both on non-permanent “baby” and on permanent “adult” teeth. It adheres to the surface of teeth, gum tissues, dental restoration, and even to the tongue. Studies have shown that plaque forms very soon, as fast as four hours after removal of the previous portion of residual plaque. When oral hygiene is poor, within about three weeks the presence of the plaque results in occurrence of gingivitis. As noted, with diligent flossing and tooth brushing gingivitis is reversible and the inflammatory conditions usually disappear. In the absence of such oral hygiene, periodontal disease starts progressing. Plague control is the only effective method of controlling chronic periodontal disease.

People use mechanical devices to control plague. There are toothbrushes, tooth paste, interdental cleaning aids, oral irrigation devices, etc. However, these techniques have several limitations and the entire tooth surface cannot be cleaned perfectly, especially if we don't see some residual plague due to almost the same color of the teeth and the paste. Moreover, once the plague is deposited in considerable quantities, it is difficult to remove it with common mechanical methods.

Numerous toothpastes are represented in the relevant art. For example, there is known U.S. Pat. No. 4,181,712 issued to Rialdi, which is directed towards toothpaste. The toothpaste composition taught 4i that patent includes, in combination with detergent base, a mixture of micronized colloidal silica gel and activated charcoal. Thanks to the absorbing properties of the charcoal that may adsorb the fermentation products and the acidity from the sugars, this formulation acts as cleaning and whitening agent and prevents caries. The paste according to Rialdi does not contain abrasive agents. Glycyrrhizin can be added as a flavoring as well as antimicrobial agents.

A published patent application US 2005/0147569 by Harwood generally relates to compositions and methods useful for oral hygiene and more particularly to compositions and methods that are useful for cleaning tongues and throats of warm-blooded animals and humans. Aqueous slurries for, cleaning the tongue and throat are comprised of finely divided charcoal, water and alcohol biocide optionally in the form of mixture. More specific embodiments of liquid portions of the aqueous slurry contains biocide selected from chloride dioxide, cetyl peridinium (CPC), Zynk Chloride, Alcohol, Hydrogen Perozxide, Triclosan, or mixture of thereof A more particular embodiment is a commercially available mouth rinse combined and mixed with some quantity of charcoal. This invention includes different methods of cleaning and using cotton tipped picks, cotton swabs and tongue scrapers.

Of further interest is a US published patent application US 20007/0092455 by Harwood, which discloses aqueous slurries useful for cleaning teeth and methods related thereto (picking and scraping). Aqueous slurries are considered to be capable of removing not only plaque and stains, but also calculus. In other aspects, US 20007/0092455 is also directed to developing methods of cleaning teeth of humans and warm-blooded animals. The slurry, which may be used to remove plaque from human or animal teeth, combines finely divided charcoal, water, alcohol, ammonia or hydrogen peroxide. Aqueous solutions of hydrogen peroxide are prepared by methods well known in the art and are used for bleaching, oxidizing, deodorizing and disinfecting. But the slurries, described in that patent application, are not constant and do not include fluoride, a system for restoration and prevention of decay, and has a burning effect due to alcohol, ammonia, or hydrogen peroxide. Besides, not everybody is motivated highly enough not only to brush and floss teeth, but also to scrape and pick to remove the calculus. The author of US 20007/0092455 considers his method suitable and effective for removing at least some calculus as part of personal oral hygiene regimen, and there are various compositions that can be used with otherwise conventional brushing methods.

Another U.S. Pat. No. 6,340,447 issued to Johnson is directed towards only methods of using odor eliminating items for hunting. Personal cleaning items utilized in the method of removing odors from a person include mouthwash and soap, which include activated carbon or activated charcoal. This toothpaste formulation is directed to particular users only (for hunters), and is produced without adding scent and flavor.

In addition, US patent U.S. Pat. No. 6,432,388 issued to Alvarez Hernandez describes whitening anti-plaque and anti-tartar low abrasivity toothpaste. The paste includes 0.1-1% papain, 5-10% xylitol, 16-18% of an abrasive system based on silica, a buffer consisting of tetrapotassium pyrophosphate and potassium dihydrogen phosphate [PH7 approximately] along with water, binding agents, aromatizing agents, coloring agents, preservatives, sweeteners, humectants, lubricants, opacifiers, re-mineralizing agents and vitamins A, B5, C, E or mixture thereof. Vitamin E is a known anti-oxidant, but the Grape Seed Oil, proposed by the writer in Black Pearl Toothpaste, is more than 50 times stronger than vitamin E and 20 times stronger than vitamin C. Grape Seed Oil also fastens healing of wounds because proanthocyanidins that the oil contains induce vascular endothelial growth, and is used for conditions related to poor circulation and swelling in case of periodontitis. This toothpaste has also anti-inflammatory and anti-cavity properties, but doesn't contain charcoal for the controlled oral hygiene. However, charcoal perfectly removes halitosis and interferes with metabolism of bacteria. Besides, charcoal breaks the adherence of microbes to the surface of the teeth and constitutes a very good delivery system for chemotherapeutic agents.

Yet, another US patent U.S. Pat. No. 6,610,277 issued to Zuckerman is directed towards appetite suppressing toothpaste. The toothpaste composition includes toothpaste based ingredients and different appetite depressing herbs. The toothpaste base ingredients include Glycerin, Sorbitol, Hydrated silica, Xylitol, Sodiun Lauryl Sulfate, carrageenan, Titanium Dioxide and flavoring agent. The appetite suppressing herbs include at least one of Garcinia Cambogia Gymnema Sylvestre, Kola Nut, citrus Aurantium, Verba mate, and Griffonia Simplicifolia. Other components include at least one of Guarana, Green Tea, Mirrh, guggul lipid and Black Seed oil. Although these kinds of toothpastes perform intraoral cleaning, they have other goals and do not promote perfect brushing and flossing to remove toxins from oral cavities.

Of further interest is U.S. Pat. No. 4,292,306 issued to Faunce. He invented a coated fluoride and a stannous fluoride containing dentifrice in the form of gel along with sodium dihydrogen phosphate, calcium pyrophosphate, glycerin, sodium carboxymethilcellulose, xylitol, distilled water and spearmint oil. The dentifrice, however, should be used preferably in the form of gel. The U.S. Pat. No. 4,292,306 teaches topical and systemic fluoride treatment. During topical fluoride treatment stannous fluoride and sodium dihydrogen phosphate provides complex stannous fluoride fluorophosphates compound at the energized tooth surface that develops a caries inhibition fluorophosphates complex. In the systemic treatment, however, the fluoride is absorbed by the mucous of the stomach as the ingestible dentifrice containing fluoride is swallowed and fluoride is introduced into bloodstream. The dentifrice, that the toothpaste contains, provides fighting against decay, but does not control oral hygiene and does not contain antioxidants and therefore does not work enough to prevent gum diseases and healing of tissues.

Another U.S. Pat. No. 4,291,017 is issued to Beierle et. al. It provides a method for reducing the cohesiveness of bacterial plaque, the adherence of plaque to teeth, and a dental composition therefor. The composition, proposed in that patent, includes Ethyl alcohol, titanium tetrafluoride, Xylitol or saccharin, NaCl, surfactant flavoring agent, coloring agent, glycerin and water. The percentage of the ingredients changes depending on if the paste is used by a dentist or a patient. Usually dentifrice is an Ethyl alcohol solution and is not recommended for children. But we know that it is very important to prevent decay in children's teeth in post-eruptive period approximately 2 years in length, during which enamel calcification continues.

Further, a Korean patent publication KR 20010011879 by Hong Seong Ho is directed towards a method for preparation of charcoal toothpaste. Toothpaste constituents such as calcium carbonate, sodium phosphate fluoride, animocapronic acid, alantoin chlorohydroxyaluminium, vitamin E and xylitol are mixed with charcoal powder. Vitamin E is a known anti-oxidant, but the Grape Seed Oil, proposed by the instant inventor of Black Pearl Toothpaste, is more than 50 times stronger than vitamin E. The Grape Seed Oil also accelerates healing of wounds because proanthocyanidins that the oil contains induce vascular endothelial growth, and is used for conditions related to poor circulation and swelling in case of periodontitis. Only part of the publication (abstract) is available in English, which part has no indication that the toothpaste of KR 20010011879 contains fluoride.

A Japanese patent publication JP 2002348223 is known that lists Itaya Yasushi and Nakajima Yuuga as owners. It is also charcoal-based toothpaste. The toothpaste contains charcoal powder, xylitol, propolis, Japanese honeysuckle, Mongolian Dandelion, tea extract, Ginseng Extract. Although the toothpaste of JP 2002348223 prevents decay and periodontitis and removes Halitosis, fluoride components were not found in the abstract of JP 2002348223 available in English.

Hoagland and Ronald, in their published patent application US200400018155, introduce a toothpaste formulation that provides hunters with the ability to practice their normal tooth brushing routine, but without adding scent. An embodiment comprises a mixture of calcium carbonate, polyol, water, sodium bicarbonate, sodium lauryl sulfate, titanium dioxide, guar gum, sodium saccharin. The tooth paste formulation of US200400018155 is intended to be used primarily by hunters. It is impossible to introduce the toothpaste of Hoagland and Ronald into a normal hygienic routine because most of the customers prefer freshness and flavors in the mouths.

Another U.S. Pat. No. 1,716,035, issued to Donchi, discloses using a combination of an activated vegetable carbon material in conjunction with Keiselguhr (an absorbent agent) in a toothpaste, or a toothpowder formulation that contains sodium bicarbonate, saccharin, flavoring such as oil of wintergreen and oil of peppermint. The Donchi's formulation has a goal just to eliminate the odor in the mouth, while the Black Pearl Toothpaste of the instant invention provides prevention of decay, prevention and control of dental plaque, gingivitis and periodontitis along with control of malodor.

### AIMS AND BRIEF DESCRIPTION OF THE INVENTIVE PRODUCT

The primary aim of the instant invention, herein also called ‘Black Pearl Toothpaste’, is to provide a synergistic composition for toothpaste to prevent decay, to prevent and control dental plague and gingivitis, and to suppress growth of wide spectrum of micro organisms that cause periodontal disease, while not exhibiting any harmful particular side effect. The inventive toothpaste should absorb and remove odor components out of the oral cavity for a long period of time, remove stain from the teeth, and polish them. The invention should allow a consumer for controlling his/her oral hygiene. Other aims of the invention can become apparent to those skilled in the art upon learning the present disclosure.

Therefore, one of objectives of the inventive tooth paste is to provide self-controlling oral hygiene. Another objective is to provide a tooth paste formulation with no side effects and free from any toxicity or toxic residue and irritation when regularly used, which tooth paste should give effective protection to teeth, restore insipient lesions of enamel. It is cosmetically acceptable and will not leave stains on the teeth. Yet another objective of the present invention is to provide formulation which contains natural and medicinal plants useful for gums and teeth which are safe, nontoxic and digestible.

In the oral care field, bad breath is rated by consumer as one of the most frequent oral problems. In most of the cases, this problem originates from the mouth cavity due to either poor oral hygiene, or from food odor, or from microbial metabolism of food and/or oral infections. This problem should be solved by the dentifrice. The inventive formulation of toothpaste also solves the problem with somewhat unpleasant burning effect that many dentifrices often have on the sensitive tissue of the mouth, tongue and lips. This burning effect in the mouth may vary from slight effects to, at time, acute burning sensation. It is one of the reasons why some people resist tooth brushing, a function badly needed by all persons in order to preserve their teeth.

The inventive composition contains agglomerates of nontoxic pigment particles, which color the plague in black or heavy dark colors and are distinctly visible on the consumer's white teeth. That makes the consumer brushing the teeth until they are completely and perfectly cleaned. The inventive toothpaste is suitable for cleaning salivary protein plaque by providing a whitening effect on the teeth surfaces. Besides, the inventive toothpaste has fungicide and tonic effects, leaves the pleasant feeling of freshness.

The inventive toothpaste comprises xylitol, sodium dodecyl sulfate (detergent), hydrated silica, water, humectants, re-mineralizing agents, binding agents, aromatizing agents, sweeteners, preservatives, charcoal, and antioxidants.

### DETAIL DESCRIPTION OF PREFERRED EMBODIMENTS OF THE INVENTION

While the invention may be susceptible to embodiment in different forms, there will be described in detail herein, specific embodiments of the instant invention, with the understanding that the present disclosure is to be considered an exemplification of the principles of the invention, and is not intended to limit the invention to that as illustrated and described herein.

### Black Pearl Toothpaste—Preferred Composition

The inventive toothpaste is suitable for the controlled oral hygiene which is provided by the colored substance, namely: charcoal. This makes the toothpaste especially useful for home oral hygiene. The paste possesses anti-oxidant property that provides healing of damaged and stressed tissue. It also possesses the absorbent property of charcoal which removes malodor from the oral cavity.

One of the base components of the inventive formulation is charcoal. Not only charcoal does adsorb impurities but it also reduces acidity by adsorbing lactic and acetic acids as waste products. The uniformly colored particles of insoluble colored material that act as a pigment are dispersed evenly in the toothpaste. Particles of carbon (or charcoal powder 0.2-0.3 in size) are suspended in the toothpaste which allows the particles to be mechanically transported to and away from the tooth surfaces and tongue. The color of the charcoal has been used to identify the particles of the residual plaque. The paste colors the plaque in distinctly visible black or dark-brown color, and makes the consumer brushing the teeth until they become essentially clean. Charcoal enhances the benefits of dentifrice delivery system, such as delivery of sodium monofluorophosphate, capable of both inhibiting of the softening of the enamel surface and promoting of restoration of enamel eroded by lesions. Charcoal is also a preservative agent and has nutritional effect by containing residual and organic components.

The toothpaste contains Xylitol. Xylitol exists in berries, fruit, vegetables, mushrooms, and birch wood. It is also found in oak tree, chestnut tree, larch, pine tree, Japanese cypress, olive, acacia, bamboo, and mulberry tree. Xylitol is active in several ways. On the one hand, it causes voluminous anti-decay action. As a five-carbon sugar alcohol, Xylitol cannot be digested by bacteria, unlike natural sugars or other sugar alcohols such as sorbitol that are six-carbon molecules and fermentable. Xylitol interferes with metabolism of microbes (streptococcus mutants which produce acid). After taking Xylitol, bacteria do not attach well on the surface and the amount of plaque decreases. Xylitol does not raise blood sugar. This is especially beneficial for patients with diabetes, asthma, allergies. On the other hand, it acts as a humidifier and refreshener, lessening the dehydration of the mouth. It also reduces the adhesivity of the bacterial species, streptococcus mutants, microbial agents directly related to the processes of tooth decay. It raises saliva's PH by about 7, protecting the enamel from acid PH. Calciuni and phosphate with such a PH, salt in saliva, start to precipitate into parts of enamel. This problem can be resolved with addition of Sodium bicarbonate as a supplement. Sodium bicarbonate affects natural mouth odor elimination. Xylitol also has been clinically proven to facilitate the remineralization of tooth enamel (Soderling et al).

The abrasive system of the invented formulation comprises calcium carbonate and charcoal, which work effectively but less abrasively than usual agents. They have great effectiveness for removing adhesive substances from the dental surfaces. But Calcium Carbonate action is more polishing than abrasive. The binding agents used are Xanthan and Cellulose Gum, analogous to those normally used in the manufacturing of toothpastes of such a type. The binding agents are present in the formulation both by 0.5% by the weight with respect to the total. The toothpaste is aromatized with Peppermint oil (0.9) and Menthol (1.0 by the weight with respect to total).

A basic problem of oral hygiene is bad breath, also known as halitosis. This bad breath is formed by microorganisms by decomposition of food residuos and dead cells of the mucous membrane. The colonization by gram-positive and gram-negative bacteria, mycobionts, and/or protozoa is responsible for bad breath or halitosis and is reported to be the primary source of gaseous volatile sulfur compounds. In the related art literature, anaerobic gram-negative bacteria are named as the causative agents (see for example “Bad Breath—Amultidisciplinary approach” Eds D. van Steenberghe and M. Rosenberg, Leuven University Press, Leuven 1996:11-121) of the halitosis. Since social intercourse is often adversely affected by bad breath, there is a great. interest in removing and preventing it. Gram-negative organisms belong to genera Bacteroides, Fusobacterium, Haemophilus, Neisseria, Porphyromonos, Prevotella, Treponema and Veillonella. Gram-positive bacteria are members of the genera Actinomyces, Eubacterium, Lactovbecillus, Staphylococcus, and Streptococcus. Pathogenic and possible pathogenic organisms belong to the group of yeasts of the Candida species (e.g. Candida Albicans). One of the objects of the presented invention is to provide effective compounds and agents fighting against the bad breath and against the microorganisms involved in the formulation of bad breath.

The inventive toothpaste also includes Camifora Myrrh, which got its name from the Arabic word “murr” (bitter). It is used in aromatherapy for its healing, purifying and uplifting characteristics. In the past, myrrh was used by many cultures for religious ceremonies and as a healing agent. The myrrh oil's healing characteristics are that it is an antiseptic, deodorant, stimulant, fungicide, and tonic. Myrrh has high antiseptic and anti-inflammatory properties. It was used to clean wounds and prevent infection.

The preservatives in the formulation are Sodium Methylparaben at the amount of 0.9 with respect to total. Methylparaben is used instead of Sodium Benzoate because there has been concerns that benzoic acid and its salts may react with antioxidants and ascorbic acid (vitamin C) forming benzene which is carcinogenic. (Goldstein)

In addition, as a sweetener, Sodium Saccharine is present in the formulation at the amount of 0.4 with respect to total. The humectant agents used to prevent dehydration and hardening of the toothpaste are Glycerin and Xylitol. Glycerin has been shown to protect against DNA damage induced by tumor promoters, ultraviolet lights and radiation, presumably via free radical scavenging. Glycerin is present in the preferred formulation at the amount of 10% with respect to total.

As a pacifier, Titanium Dioxide can be used, which is present in the preferred embodiment of the inventive formulation in the amount of 0.5 G with respect to the total. As a remineralizing agent, Sodium Monofluorophosphate (MFP) may be present at the amount of 0.76-1.1% by weight with respect to the total. Sodium monofluorophosphate has been approved for use in dentifrices: this compound has the empirical formula: Na₂ PO₃F. The mechanism of action of MFP involves a chemical reaction with the surface enamel and serves to make that surface more resistant to demineralization (inhibiting of the softening of the enamel and promoting restoration of enamel eroded by lesions). The use of sodium monofluorophosphate in a dentifrice contributed significantly to the control of dental caries.

A unique and interesting characteristic of MFP is its compatibility with dentifrice abrasive system. The fluoride in MFP remains largely complexed as PO₃F₅ in solution and continues providing cariostatic activity. The clinical trial has shown that in dentifrices employing silica-abrasive system and sodium monofluorophospate as the fluoride source, 1,500 ppm of fluoride is statistically significant more effective than 1,000 ppm with a margin of superiority of above 15%. The presence of fluoride prevents accumulation of bacteria of the teeth. Charcoal and Calcium Carbonate enhance the delivery of Sodium Monofluorophosphate.

When bacteria come into contact with gums, they create toxins and break down the gum lining, creating tiny ulcers. Penetrating into the deepest layers of the gums, they turn into anaerobic bacteria that appear to cause the most damage. In one invasive form neutrophils in the inflamed tissue have trouble homing on bacteria, throwing on them chemical grenades called superoxides. Oxidative stress (increased production of free radical oxygen species) has been shown to result from excessive production by hyperresponsive neutrophils. Oxygen-free radicals are injurious to all classes of biologically important molecules and are associated with chronic inflammatory conditions including periodontitis. But instead of destroying bacteria, they destroy the periodontal ligaments, which anchor the teeth to the bones. The oxidative damages are associated with cellular respiration. Respiration is intercellular oxidation and may be anaerobic and aerobic. Free radicals generated during the phagocitosis and neutrophil activation though an intend to kill bacteria or damaged tissue, can also kill the body's own immune cells. Antioxidants reduce this self-destruction. Therefore, the inventive toothpaste contains Grape Seed Oil and Extract of Brazilian Acai Berry, which are great sources of OPCs (oligomeric proanthocyanidins). OPCs are known to be strong antioxidants.

Grape Seed Oil is a natural plant ingredient (bioflavonoid) which strengthens and protects living tissue. The antioxidant property in Grape Seed Oil is 50 times stronger than that of Vitamin E and 20 times stronger than that of Vitamin C. The commercial opportunity of extracting grape seed ingredients has been known as obtaining strong antioxidants. Preliminary research shows that the grape seed extract may have other possible anti-disease properties, such as in laboratory models. There are wound healing grape seed proanticyanidins induced the vascular endothelial growth factor and accelerated healing of injured skin on mice. It may help preventing decay: seed phenolics may inhibit oral sugar metabolism and the retard growth of certain bacteria causing dental decay. The grape seed extract helps fighting against osteoporosis by enhancing bone density. Also, proanticianidins may protect against carcinogenesis and provide supplementation for sunscreen protection. The grape seed extract is used for conditions related to poor blood circulation, when the gum is swelling, which could be caused by injury, surgery, or inflammation. The grape seed extract strengthens the capillaries, insuring a good supply of oxygen and nutrients. Various formulations of grape seed are used internally (orally) and externally (topically).

The inventive formulation also includes another antioxidant, the acai berry extract. The acai berry is a member of genus Euterpe which contains 8 species of palms native to tropical Central and South America, growing mainly in floodplains and swamps. The acai berry has been around for thousands of years and not until the 1990's was it introduced to the Western world. The acai berry was found to possess tremendous health property. It was first used by the tribes of the Amazon jungle as a cure for various ailments. The powder of berry was shown to contain vitamin C, calcium, iron, vitamin A, as well as aspartic acid, glutamic acid and amino acid. The acai berry was discovered to have natural antioxidant properties. The tribes of Amazon knew of these properties and found out that they could help to build the immune system and fight infection. The acai berry is also used to produce an antibiotic that helps to fight against staphylococcus aureus. It is very interesting that the berry has a vibrant taste with hint of chocolate. It is rich in proteins, vitamin E, minerals and essential Omega oils. Known as a miracle fruit, acai berry is five times more potent than gingko biloba, a commonly used herbal therapy product. Acai is naturally low in sugar and its flavor is described as a mixture of red wine and chocolate (Susan Talcott).

Thusly, the inventive toothpaste includes the following components: Sodium monofluorophosphate, Glycerin, Water, Calcium carbonate, Xylitol-70%, Sodium dodecyl sulfate, Sodium Saccharin, Sodium Bicarbonate, Sodium methylparaben, Xanthan Gum (E-415), Cellulose Gum (E-466), Hydrated Silica, Titanium dioxide (E-171), Peppermint oil, Commiphora myrrh Extract, Menthol, Charcoal (vegetable), Grape Seed Oil, and Extract of Brazilian Acai Berry.

In a preferred embodiment, Black Pearl Toothpaste comprises the above enumerated ingredients with weight percentage amounts listed in the following Table:

| ## | Ingredients | %% with respect of the total |
|---|---|---|
| 1 | Sodium monofluorophosphate | 0.76-1.1 |
| 2 | Glycerin | 10.0 |
| 3 | Water | 30 |
| 4 | Calcium carbonate | 20.0 |
| 5 | Xylitol-70% | 10.0 |
| 6 | Sodium dodecyl sulfate | 0.9 |
| 7 | Sodium Saccharin | 0.4 |
| 8 | Sodium Bicarbonate | 0.5 |
| 9 | Sodium methylparaben | 0.9 |
| 10 | Xanthan Gum (E-415) | 0.5 |
| 11 | Cellulose Gum (E-466) | 0.5 |
| 12 | Hydrated Silica | 7.0 |
| 13 | Titanium dioxide (E-171) | 0.5 |
| 14 | Peppermint oil | 0.9 |
| 15 | Commiphora myrrh Extract | 0.5-0.7 |
| 16 | Menthol | 0.5-1.0 |
| 17 | Charcoal (vegetable) | 8.0 |
| 18 | Grape Seed Oil | 3 |
| 19 | Extract of Brazilian Ocai Berry | 3 |

### Black Pearl Toothpaste—Method of Preparation

The toothpaste provided by this invention can be easily prepared by mixing the above-indicated components in suitable quantities according to a desired volume of toothpaste, and by stirring the mixture at a temperature that does not lead to degradation and thermal denaturizing.

A sample method for preparation of 100 weight units of the inventive toothpaste comprises the steps of:
a) Dissolve 7 weight units of pure Xylitol in 10 weight units of water using heat application within a predetermined temperature range to avoid degradation and thermal denaturizing, to speed the reaction of dissolution. Measure 10 weight units of the product for use in the further steps, discard the remainder.
b) Dissolve Sodium Dodecyl Sulfate, Sodium Saccharin, Sodium Bicarbonate in 20 weight units of water, gently mixing to avoid bubbling of Sodium Dodecyl.
c) Dissolve Sodium Methylparaben in the solution obtained in Step (b).
d) Add both gums (Xanthan Gum and Celllulose Gum) in the solution obtained in Step (a) while mixing vigorously until it thickens.
e) Add the solution obtained in Step (d) to the solution obtained in Step (c) in parts while mixing well.
f) Add to the mixture Peppermint Oil, Commiphora Myrrh Extract, Grape Seed Oil and Acai Berry while mixing well.
g) Mix thoroughly the mixture with Calcium Carbonate to prepare the paste.
h) Mix Sodium Monofluorophosphate, Titanium Dioxide and Menthol (0.5%) thoroughly.
i) Levigate Charcoal with Glycerin and mix with the paste.
j) Levigate silica with the paste.
k) Mix all the components thoroughly.
l) Pack the paste.

### Method for Preparation of the Menthol Powder Using Crystals

The Menthol powder for the step (h) above can be preliminary prepared in the following sub-steps:
h-1) weighting a predetermined amount of Menthol and dissolving it in Alcohol obtaining a resultant mixture; and
h-2) triturating the resultant mixture until Alcohol evaporates leaving powdered Menthol.

The preparation of the inventive toothpaste is carried out at a predetermined temperature that does not allow the components to degrade or denaturate.

The following references have also been used in the present disclosure:
Nassarh, Kantarci A, van Dyke T E, Diabetic periodontitis: Model fro activated innate immunity and impaired resolution of inflammation, periodontal 2000 2007; 43:233-44
Heitz-Mayfield Ld Schatzle M Loe H et al clinical cource of chronic periodontitis. II Incidence, characteristics nad time of ovvurrence of initial periodontal lesion J Klin periodontal 2003; 30(10):902-908
Schatzle M, Loe H, Burgin w. et al. Clinical course of chronic periodontitis I Role of gingivitis j Clin periodontal 2003, 30 (10); 887-901
Loe H, Brown L. J. Early onset periodontitis in the united states of America J periodontal 1991; 62 (10): 608-618
Kantarci A, Van Dyke T E Resolutin of inflammation in periodontitis [Review] Periodontal 2005; 76 (11 Suppl); 2168-2174
̂Plotkin M J balick M J (Apr. 1984) “Medical used of South American palms.” JEthnopharmacol 10(2):157-79 pmid 6727398 9http://www.nebi.nlm.nih.gov/pubmed/6727398)
̂abcd Schauss A G, WUX, Prior RL, OuB, Huang D, Ownes J, Agarwal A, Jensen G S, Hart A N, Shanbron E. (2006) “Antioxidant capacity and other bioactivities of the freeze-dried Amazonian palm berry, Euterpe oleraceae Mart. (acai)”J. Agric Food Chem 54 (22):8604-10. PMID 17061840 (http:/www.ncbi.nlm.nih.gov/pubmed/17061840)
̂Kuskoski E. M., Asuero A. G., Morales M. T., Felt R. (2006) “Wild Fruits and Pulps of Frozen Fruits: antioxidant activity, polyphenols and anthocyanins (http://www.scielo.br/scielo.php?script=sci_arttext&pid-S0103-84782006000400037&|ng=en&nrm=iso)” Ciene rural 36 (4 July/August). http://www.scielo.br/scielo.php?script=sci_arttext&pid=S0103-84782006000400037&|ng=en&nrm=iso.
̂ab Lichtenthaler R, Rodrigues R. B., Maia J. G., Papagiannopoulos M., Fabricius H., Marx F. (February 2005). “Total oxidant scavenging capacities of Euterpe Oleracea Mart. (acai) fruits.” Int J. Food Sci Nutr 56 (1): 53-64 doi:10.1080/09637480500082082 (http://dx.doi.org/10.1080/09637480500083082). PMID 16019315 (http://www.ncbi.nlm.nih.gov/pubmed/16019315)

I claim:

1. A toothpaste comprising the following components: Sodium Monofluorophosphate, Glycerin, Water, Calcium Carbonate, Xylitol-70%, Sodium Dodecyl Sulfate, Sodium Saccharin, Sodium Bicarbonate, Sodium Methylparaben, Xanthan Gum (E-415), Cellulose Gum (E-466), Hydrated Silica, Titanium Dioxide (E-171), Peppermint Oil, Commiphora Myrrh Extract, Menthol, Charcoal, Grape Seed Oil, and Extract of Brazilian Acai Berry.

2. The toothpaste according to claim 1, wherein said components have the following weight percentages of the total 100%:
Sodium Monofluorophosphate: 0.76-1.1%, Glycerin: 10.0%, Water: 30%, Calcium Carbonate: 20.0%, Xylitol-70%: 10.0%, Sodium Dodecyl Sulfate: 0.9%, Sodium Saccharin: 0.4%, Sodium Bicarbonate: 0.5%, Sodium Methylparaben: 0.9%, Xanthan Gum (E-415): 0.5%, Cellulose Gum (E-466): 0.5%, Hydrated Silica: 7.0%, Titanium Dioxide (E-171): 0.5%, Peppermint Oil:. 0.9%, Commiphora Myrrh Extract: 0.5-0.7%, Menthol: 0.5-1.0%, Charcoal: 8.0%, Grape Seed Oil: 3%, and Extract of Brazilian Acai Berry: 3%.

3. A method of preparation of the toothpaste according to claim 1, said method comprising the steps of:
a) dissolving 7 weight units of pure Xylitol in 10 weight units of water using heat application within a predetermined temperature range to avoid degradation and thermal denaturizing, to speed the reaction of dissolution, measuring and allocating 10 weight units of the product for use in the further steps;
b) dissolving Sodium Dodecyl Sulfate, Sodium Saccharin, Sodium Bicarbonate in 20 weight units of water, gently mixing to avoid bubbling of Sodium Dodecyl;
c) dissolving Sodium Methylparaben in the solution obtained in Step (b);
d) adding both gums Xanthan Gum and Celllulose Gum in the solution obtained in Step (a) while mixing vigorously until it thickens;
e) adding the solution obtained in Step (d) to the solution obtained in Step (c) in parts while thoroughly mixing;
f) adding to the mixture Peppermint Oil, Commiphora Myrrh Extract, Grape Seed Oil and Acai Berry while thoroughly mixing;
g) mixing thoroughly the mixture obtained in Step (f) with Calcium Carbonate to prepare said toothpaste;
h) mixing Sodium Monofluorophosphate, Titanium Dioxide, and Menthol (0.5%) thoroughly;
i) levigating Charcoal with Glycerin and mixing with the toothpaste;
j) levigating Silica with the toothpaste;
k) mixing the mixture obtained in Step (j) thoroughly; and
l) packing the mixture obtained in Step (k).

4. The method of claim 3, wherein Menthol of Step (h) is preliminary obtained following the sub-steps of:
h-1) weighting a predetermined amount of Menthol and dissolving it in Alcohol obtaining a resultant mixture; and
h-2) triturating the resultant mixture until Alcohol evaporates leaving powdered Menthol.
